(19) United States
(12) Patent Application Publication
(10) Pub. No.: US 2007/0232900 A1
(43) Pub. Date: Oct. 4, 2007

(54) Medical navigation and positioning system containing an operation system and method for operation

(75) Inventor: Martin Hoheisel, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/732,449

(22) Filed: Apr. 3, 2007

(30) Foreign Application Priority Data
Apr. 3, 2006 (DE) 10 2006 015 349.9

(51) Int. Cl. A61B 5/05

(52) U.S. Cl. 600/424

(57) ABSTRACT

For flexible and hygienic operation, a medical navigation and positioning system with an operation system is provided for assignment to a medical imaging device, with the navigation and positioning system featuring a positioning facility, a processing unit and an operating instrument, with the position of the operating instrument being able to be located by means of the positioning facility, with the processing unit being embodied for checking the current position of the operating instrument in relation to predetermined positions and, if the position of the operating instrument matches a predetermined position, at least one corresponding operating function of the operation system or of the medical device assigned to the predetermined position being able to be initiated.

### CROSS REFERENCE TO RELATED APPLICATIONS

This application claims priority of German application No. 10 2006 015 349.99 filed Apr. 3, 2006, which is incorporated by reference herein in its entirety.

### FIELD OF THE INVENTION

The invention relates to a medical navigation and positioning system, containing an operation system for assignment to a medical imaging device and a method for operation of said medical navigation and positioning system or said medical imaging device.

### BACKGROUND OF THE INVENTION

Navigation systems are frequently used in medical surgical procedures and interventions to provide physicians with image-based support for handling their instruments, for example puncturing needles or catheters. One example of this type of navigation and positioning system provided for medical applications is the CAPPA system from CAS Innovations AG (http://www.cas-innovations.de/).

In general a medical navigation and positioning system consists of a positioning system, a control computer and a display unit. The positioning system operates in determining the position of an object, optically or electromagnetically for example; examples of such positioning systems are the Aurora or Polaris systems from Northern digital Inc. (NDI, http://www.ndigital.com/). The control computer spatially relates the position of the located instruments to a three-dimensional 3D image data set, which for example can be supplied by an imaging device, so that a superposed image can be shown on the display unit.

Such a system is normally operated using a standard keyboard or mouse or by means of a special operator console. The CAPPA system for example has a touchscreen on which different functions are initiated by touching marked fields on the screen. The disadvantage of the existing operation systems lies in the fact that physicians operating on a patient, in order to operate the navigation system at the same time, must look away from the operation area or the display unit and must move their hand away from the sterile operation area to touch the screen or keyboard.

### SUMMARY OF THE INVENTION

The object of the invention is to create, for surgical interventions, an operation system for operating a medical navigation and positioning system or a further medical device, which allows flexible and hygienic operation.

According to the invention, the object is achieved with a medical navigation and positioning system containing an operation system and with a method for operating said system as claimed in the claims. Advantageous embodiments of the invention are the subject of the subclaims in each case.

The invention allows a simple and flexible operation of the medical navigation and positioning system or of a further medical device by assigning functions of the operation system or of the medical device to predetermined positions of an operating instrument. The invention guarantees in particular that an operator within a sterile operation area does not have to move his/her hand out of the sterile operation area. Operation within the sterile operation area is easily possible since the functions of the operation system (or the further medical device) can be initiated by manually moving the operating instrument to the predetermined position, said operating instrument already being located in the sterile operation area for example. In this way a physician involved in a medical intervention can meet hygiene standards with little effort, without for example being dependent on the assistance of an additional operator.

In accordance with an embodiment of the invention the operation system features a display unit and the display unit is embodied for displaying the operating instrument and for displaying the predetermined positions. This allows the operator to operate the system especially quickly and flexibly, since said operator does not have to move their eyes away from their field of vision during operation.

According to a further embodiment of the invention the predetermined positions are able to be displayed by real markings or elements. This provides an additional orientation aid for the operator and simplifies the operation of the medical navigation and positioning system or of the further medical device.

In an advantageous manner, for especially simple and clear operation, the predetermined positions are able to be displayed by virtual markings or elements on the display unit.

According to a further embodiment of the invention the predetermined positions can be displayed by mapping the real markings or elements on the display unit. This also allows improved clarity during operation.

According to a further embodiment of the invention the medical navigation and positioning system is mechanically integrated into the medical imaging device. This enables existing components already present in the medical imaging device such as display unit or processing unit to also be used in the medical navigation and positioning system and operation of the medical imaging device to be provided especially simply through the operation system.

According to a further embodiment of the invention the medical imaging device is embodied for 3D imaging.

### BRIEF DESCRIPTION OF THE DRAWINGS

The invention, as well as further advantageous embodiments in accordance with features of the subclaims, are explained in greater detail below with reference to schematic exemplary embodiments in the drawing, without the invention being restricted by this to these exemplary embodiments; the figures show:

FIG. 1 a medical x-ray apparatus with an externally assigned medical navigation and positioning system;

FIG. 2 a display unit with an operating instrument shown in a first position and virtual target marks included on the display;

FIG. 3 a display unit with an operating instrument shown in a second position and virtual target marks included on the display;

FIG. 4 a view of a patient from above with x-ray marks arranged on them;

FIG. 5 a display unit with target marks defined by means of the x-ray markers included on the display;

FIG. 6 a further medical x-ray apparatus with an integrated medical navigation and positioning system.

### DETAILED DESCRIPTION OF THE INVENTION

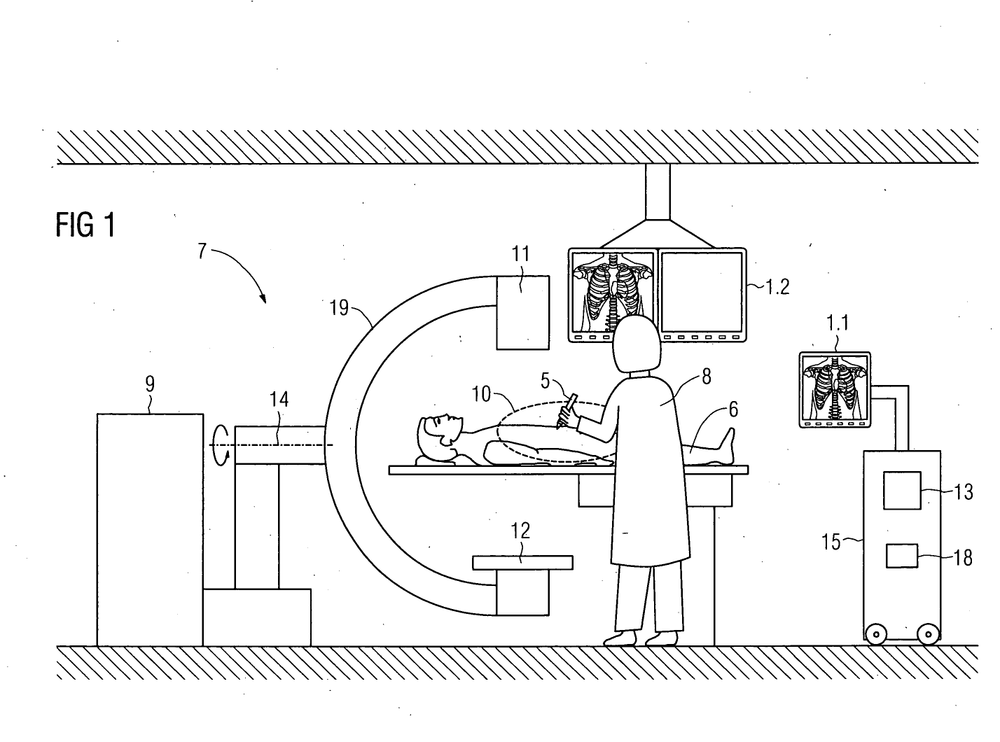

FIG. 1 shows an inventive medical navigation and positioning system 15, which is arranged externally to a medical imaging device in the form of a 3D x-ray device 7 and is assigned to the latter. The 3D x-ray device 7, in addition to a control unit 9 and a monitor unit 1.2, features a C-arm 19 with an x-ray source 11 and an x-ray detector 12 that can be rotated around at least one axis 14.

Three-dimensional x-ray images of a patient 6 or of parts of the body of the patient 6 can be recorded with a 3D x-ray device 7. This involves irradiating the patient 6 with x-ray radiation emitted by the x-ray source 11 and recording a number of 2D x-ray images at different angles of rotation of the C-arm 19, by means of the x-ray detector 12 for example. Subsequently a 3D reconstruction of the 2D x-ray images, which can be displayed on the monitor unit 1.2, is undertaken by means of the control unit 9 or by means of an image processing unit arranged within it.

The inventive medical navigation and positioning system 15 comprises a positioning facility 18, a control computer 13, an associated monitor 1.1 and an operating instrument 5. The operating instrument 5 is formed by a special pointer instrument or can also be formed by an instrument normally used by a physician 8 for an intervention into the body of the patient 6, such as a catheter or a puncturing needle.

The operating instrument 5 must be embodied such that its position is able to be located by the positioning facility 18. If the positioning facility 18 involved is an electromagnetically-operating positioning facility 18, the operating instrument 5 must for example have a coil by means of which its position is able to be established. A sensor glove can for example also be embodied as an operating instrument 5, which the physician 8 wears on his hand and by which he can operate the medical navigation and positioning system 15 in accordance with the invention.

In general, with medical interventions assisted by a known navigation and positioning system according to the prior art, at least one x-ray image is recorded before the intervention in the body of the patient 6. This 3D x-ray image is transmitted to the navigation and positioning system and displayed on its monitor. Under normal circumstances the position of an instrument of the physician 8 is located during the intervention in the body of the patient 6 using a known positioning system and spatially related by means of a control computer of the known navigation and positioning system to the 3D x-ray image and also displayed on the monitor unit. The physician 8 performing the intervention can subsequently observe the respective position of their instrument on the monitor unit. The system is normally operated by buttons arranged on the monitor unit for example or by means of a monitor embodied as a touch screen.

The inventive navigation and positioning system 15 now allows operation such that the hand of the physician 8 does not have to leave the sterile area 10. In addition the physician can perform their activities without having to look away from the monitor 1.1/the monitor unit 1.2.

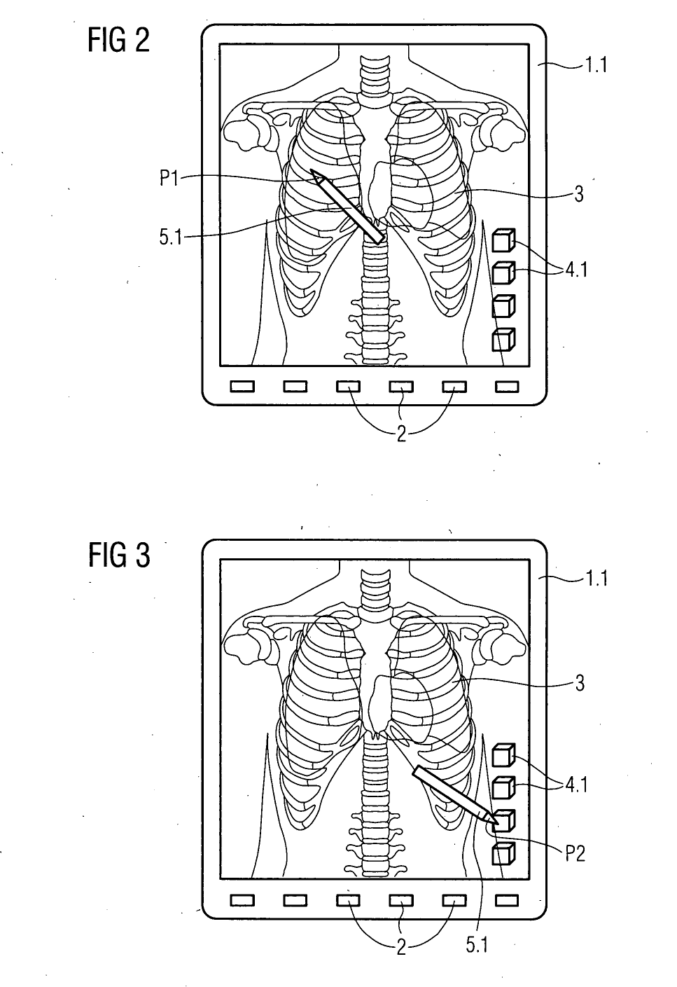

FIG. 2 shows the monitor 1.1 of the inventive navigation and positioning system 15, on which, as well as the 3D x-ray image 3 and the operating instrument image 5.1, virtual elements 4.1 are displayed. The position of the operating instrument 5 is located in this case by the positioning facility 18, related by the control computer 13 of the inventive navigation and positioning system 15 to the patient and to the 3D x-ray image 3 and shown in its correct orientation. The virtual elements 4.1 are in this case preferably included at the edge of the display of the 3D x-ray image 3 on the monitor 1.1 in order not to cover over any important parts of the 3D x-ray image.

The initiation of a function of the inventive navigation and positioning system 15 is linked to each of the virtual elements 4.1; such functions can for example be the rotation or the zooming-in or the moving of the image on the monitor 1.1 or the setting of brightness and/or contrast or other types of image processing. Supplementary functions such as the measurement of distances between different points of the image or the definition of insertion and target points can also be initiated. In the 3D x-ray device for example display functions such as zooming or movement or operating functions, such as the deployment of x-ray radiation or the movement of the C-arm 19, can also be initiated.

During the intervention the position of the operating instrument 5 is constantly located by means of the positioning facility 18 and shown on the monitor 1.1, so that the physician 8 can follow each of their actions on the monitor 1.1. FIG. 2 shows the operating instrument mapping 5.1 in a first position P1. If the physician 8 moves the operating instrument 5 manually, so that the operating instrument mapping 5.1 adopts a second position P2—shown in FIG. 3—in which the operating instrument image 5.1 touches one of the virtual elements 4.1, this initiates the operating function linked to the virtual element 4.1.

This is done by the control computer 13 of the inventive navigation and positioning system 15 checking the position of the operating instrument 5 based on the information of the positioning facility 18 and, if there is a match between the current position of the operating instrument 5 and a predetermined position such as the second position P2, initiating the actuation of the corresponding operating function linked to the position.

Ideally the virtual elements 4.1 are also shown in three dimensions, so that a three-dimensional positioning of the operating instrument 5 must be undertaken in order to initiate a function. This minimizes the danger of a function being initiated by mistake. In addition it is possible to operate the medical navigation and positioning system 15 using the virtual element 4.1 and the operating instrument 5 by means of known operating buttons 2 or a keyboard.

According to an alternate embodiment of the invention, real markings or elements are used to initiate functions of the navigation and positioning system 15. These real markings or elements are arranged such that their position is known in relation to the patient 6 or in relation to the image data of the 3D x-ray image 3. Thus the real markings or elements can be arranged, prior to the intervention for example, on the patient bed or even on the patient 6, their position detected and linked to functions. Such real markings or elements can for example be formed by buttons, colored markings or colored adhesive strips. The physicians 8 touches one of the real markings or elements with the operating instrument 5. The position of the operating instrument 5 is located using the positioning facility 18, the position is checked in relation to the real element or the marking and, if they match, the function linked to the real element or the real marking is subsequently initiated. The real elements or markings can in addition be shown in the display on the monitor 1.1.

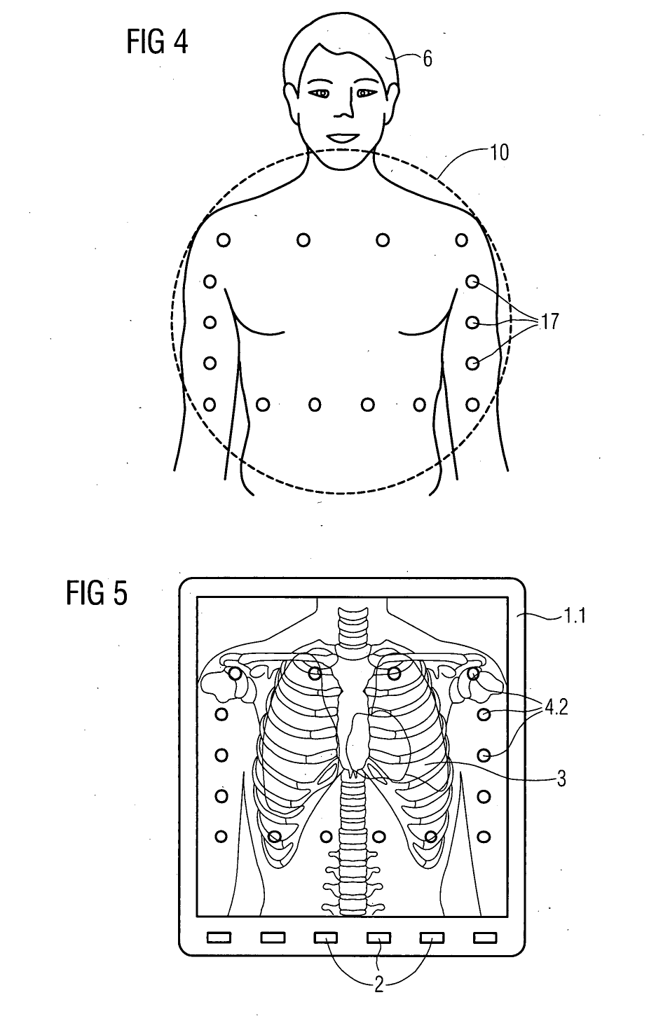

FIG. 4 shows a patient on who x-ray markers 17 are arranged for spatial assignment of the 3D x-ray image. The x-ray markers 17 are used as real elements and are—as shown in FIG. 5—displayed as element images 4.2 on the monitor 1.1. If the operating instrument 5 is now moved into a position in which the operating instrument image 5.1 coincides with an element image 4.2, the function linked to the element image 4.2 is initiated.

In addition to the functions of the medical navigation and positioning system 15, there can also be provision for initiating functions of the 3D x-ray device 7 by means of the virtual target marks 4.1 or the element images 4.2.

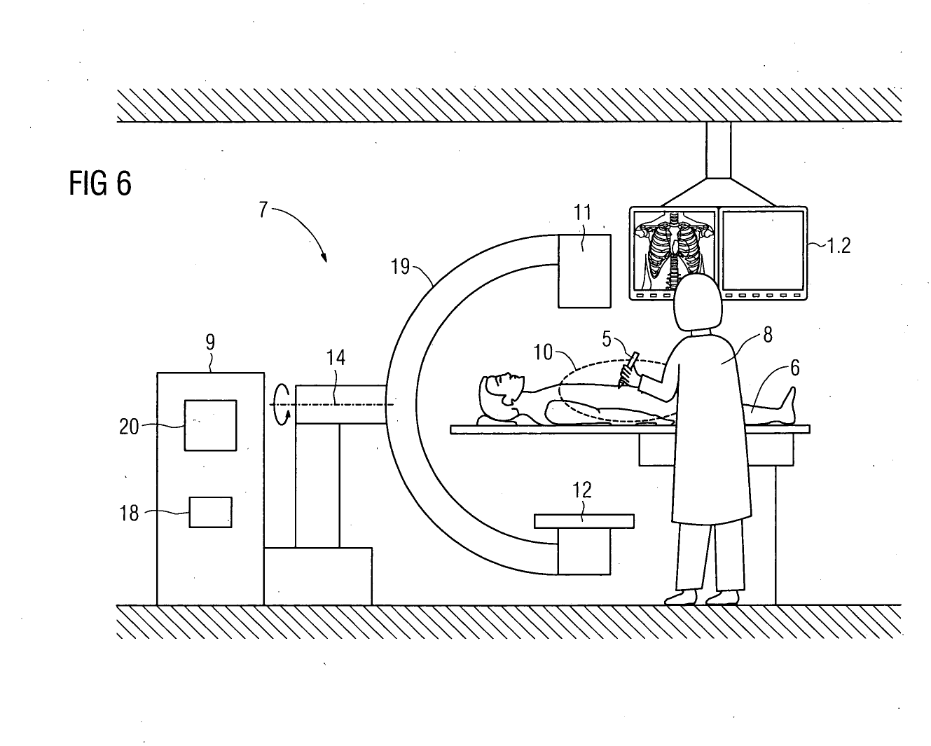

FIG. 6 shows the 3D x-ray device 7 with a mechanically-integrated inventive medical navigation and positioning system. The integrated medical navigation and positioning system features the positioning facility 18 and the operating instrument 5 and uses a control computer 20 of the 3D x-ray device 7 present in the control unit 9 to spatially relate the position of the operating instrument 5 to the 3D x-ray image 3. In addition the integrated medical navigation and positioning system uses the monitor unit 1.2 of the 3D x-ray device 7 to display the 3D x-ray image 3, the operating instrument 5 and the virtual elements 4.1 or the element mappings 4.2.

Any number of virtual elements 4.1 and thereby initiation positions of the operating instrument 5 can be defined for example before use of the medical navigation and positioning system and of the 3D x-ray device by software and linked to the desired functions. Predetermined sets of virtual elements 4.1 can also be used in a similar way to a predetermined keyboard. Real target marks can be placed in any arrangement as required before the use of the medical navigation and positioning system and of the 3D x-ray device and linked to desired functions.

The virtual elements 4.1 can in addition be displayed as a function of the relevant state of the navigation and positioning system, so that only specific functions can be initiated at a specific point in time. This is useful for example for presenting a hierarchical menu structure or for hiding functions that are critical at a particular point in time, such as an application of x-ray radiation.

The inventive medical navigation and positioning systems are especially well suited to use with medical imaging devices for 3D presentation, such as with 3D computer tomography devices, 3D magnetic resonance devices or Positron Emission Tomography devices.

Thus the invention can be summarized as follows: For flexible and hygienic operation, a medical navigation and positioning system with an operation system is provided, for assignment to a medical imaging device, with the navigation and positioning system featuring a positioning device, a processing unit and an operating instrument, with the position of the operating instrument being able to be located by means of the positioning device, with the processing unit being embodied for checking the current position of the operating instrument in relation to predetermined positions and, if the position of the operating instrument matches a predetermined position, at least one of the corresponding operating functions of the operating system or of the medical device assigned to the predetermined position being able to be initiated.

1.-12. (canceled)

13. A medical navigation and positioning system assigned to a medical imaging device, comprising:
an operating instrument that operates in a patient;
a positioning unit that locates a current position of the operating instrument in the patient; and
a processing unit that:
compares the current position of the operating instrument with a predetermined position of the operating instrument, and
activates an operating function of the navigation and positioning system assigned to the predetermined position if the current position of the operating instrument matches to the predetermined position.

14. The medical navigation and positioning system as claimed in claim 13, further comprising a display unit to display the operating instrument and the predetermined position.

15. The medical navigation and positioning system as claimed in claim 14, wherein the predetermined position is displayed on the display unit by mapping a real marking or a real element to the display unit.

16. The medical navigation and positioning system as claimed in claim 15, wherein the real marking or the real element is arranged on a patient bed or on the patient.

17. The medical navigation and positioning system as claimed in claim 15, wherein the real marking or the real element is selected from the group consisting of: a button, a colored marking, and a colored adhesive strip.

18. The medical navigation and positioning system as claimed in claim 14, wherein the predetermined position is displayed on the display unit by a virtual marking or a virtual element.

19. The medical navigation and positioning system as claimed in claim 13, wherein the medical imaging device is a 3D medical imaging device.

20. The medical navigation and positioning system as claimed in claim 19, wherein the 3D medical imaging device is a 3D x-ray device.

21. The medical navigation and positioning system as claimed in claim 13, wherein the medical navigation and positioning system is integrated into the medical imaging device.

22. The medical navigation and positioning system as claimed in claim 13, wherein an operating function of the medical imaging device assigned to the predetermined position is activated if the current position of the operating instrument matches to the predetermined position.

23. A medical imaging device with an integrated medical navigation and positioning system, comprising:
a radiation source that emits a radiation to a patient;
an imaging detector that records an image of the patient by detecting the radiation;
an operating instrument that operates in the patient;
a positioning unit that locates a current position of the operating instrument in the patient; and
a control unit that:
compares the current position of the operating instrument with a predetermined position of the operating instrument, and
activates an operating function of the medical imaging device assigned to the predetermined position if the current position of the operating instrument matches to the predetermined position.

24. The medical imaging device as claimed in claim 23, further comprising a display unit that displays the image of the patient, the operating instrument, and the predetermined position.

25. The medical imaging device as claimed in claim 24, wherein the predetermined position is displayed on the display unit by mapping a real marking or a real element arranged on a patient bed or on the patient to the display unit.

26. The medical imaging device as claimed in claim 24, wherein the predetermined position is displayed on the display unit by a virtual marking or a virtual element.

27. The medical imaging device as claimed in claim 23, wherein an operating function of the medical navigation and positioning system assigned to the predetermined position is activated if the current position of the operating instrument matches to the predetermined position.

28. A method for operating a medical navigation and positioning system assigned to a medical imaging device, comprising:
locating a current position of an operation element in a patient;
comparing the current position of the operation element with a predetermined position of the operation element; and
activating an operating function of the navigation and positioning system related to the predetermined position if the current position of the operation element matches the predetermined position.

29. The method as claimed in claim 28, wherein an operating function of the medical imaging device related to the predetermined position is activated if the current position of the operation element matches to the predetermined position.

30. The method as claimed in claim 28, wherein the operation element and the predetermined position are displayed on a display unit.

31. The method as claimed in claim 30, wherein the predetermined position is displayed on the display unit by mapping a real marking or a real element arranged on a patient bed or on the patient to the display unit.

32. The method as claimed in claim 30, wherein the predetermined position is displayed on the display unit by a virtual marking or a virtual element.
